(19) United States
(12) Patent Application Publication
(10) Pub. No.: US 2023/0326605 A1
(43) Pub. Date: Oct. 12, 2023

(54) Multi-agent automatic voting system for fast, on-the-fly imaging and diagnosis of retina diseases using case based expert systems

(71) Applicant: Sameh Mohamed Nabil Mostafa Mesrega, Shebin Elkom (EG)

(72) Inventor: Sameh Mohamed Nabil Mostafa Mesrega, Shebin Elkom (EG)

(21) Appl. No.: 18/335,160

(22) Filed: Jun. 15, 2023

(51) Int. Cl. G16H 50/20

(52) U.S. Cl. CPC G16H 50/20

(57) ABSTRACT

The identification of retinal disorders can be made by spotting the existence of deformation associated with the disease. Although clinicians may be able to diagnose the disease through vascular abnormalities, its resource demands are high. This invention increases reliability of retina disease diagnosis through facilitating collaboration between human- and machine-agents using an electronic voting system, wherein agents use Case Based Reasoning and simplified, but accurate, easy to use retina imaging devices to reach the best possible medical judgment. The inventive solution to this problem is specified in the Claims.

### TECHNICAL FIELD

According to WHO 80% of vision disability can be avoided if detected at an early stage. They estimate that more than 360 million people will be affected worldwide by diabetes mellitus by 2030. All those people will be at risk of developing diabetic macular edema. With such numbers, detecting retinal disorders manually by ophthalmologist becomes a difficult task. The identification of retinal disorders can be made by spotting the existence of deformation associated with the disease. Although clinicians may be able to diagnose the disease through vascular abnormalities, its resource demands are high. In underdeveloped countries where the diabetic population is high, this equipment may not be readily accessible. The major challenges are mass screening, skilled technician, observer variability and, early detection [Rajagopalan N, N V, Josephraj A N, E S. Diagnosis of retinal disorders from Optical Coherence Tomography images using CNN. PLoS One. 2021 Jul. 27; 16(7)]. While electronic decision support systems provide more efficient and reliable alternatives, expert human intervention in the diagnosis process is indispensable to avoid occasionally occurring machine-mistakes and/or compensate for insufficient data.

In this invention a multi-agent, voting based decision support system for detecting retinal disorders is described. In this system, human- and machine-agents collaborate in a seamless way to reach a final diagnosis: Agents are assisted by simple imaging devices, taking on-the-fly pictures of the retina. Machine decisions are based upon case-based expert system analysis. The voting system and methods comprises, among other steps, collecting votes of human- and/or machine-agents in voting sessions dedicated to specific diagnosis suggestions. Communications to and from an agent are automatically initiated in accordance with the allocation of a Voting Manager (FIG. 1). Beyond managing the voting process: The Communications System responsibility is to send and receive retina images and associated data from and to repositories of all agents. Those repositories contain three main components as well as their associated applications: Case Databases, Medical Databases and Image Processing databases.

### PRIOR ART

Known are digital retina imaging devices like in [U.S. Pat. No. 7,331,669B2], where a portable, lightweight digital imaging device uses a slit scanning arrangement to obtain an image of the eye, in particular the retina. The scanning arrangement reduces the amount of target area illuminated at a time, thereby reducing the amount of unwanted light scatter and providing a higher contrast image. In [U.S. Pat. No. 7,338,167] a retinal imaging system includes a light source and optics which receive light from the light source and which transmit the light to produce a beam that is substantially convergent. The beam penetrates a lens of an eye and diverges following penetration of the lens to illuminate an area of a retina of the eye. An imaging device receives a reflection of light from the retina. Also: [U.S. Pat. No. 9,398,851] describes a retinal imaging device and illumination module. The illumination module includes a light source to provide an incident beam along an illumination axis, a condenser lens at a spaced apart distance from the light source for condensing the incident beam and emanating a condensed incident beam, a transparent plate at a spaced apart distance from the condenser lens, wherein the transparent plate comprises a light absorber, at least one projections lens system to focus the condensed incident beam, a shield at a spaced apart distance from the projection lens system to yield a first partially blocked beam to form a cornea illumination doughnut, and a perforated mirror comprising a hollow cylinder. Drawback in all three previous examples is that no A.I. and/or digital decision support exists.

Known are automatic retina follow up system through images as described in [U.S. Pat. No. 9,462,945B1], for example. There: Systems and methods of obtaining and recording fundus images by minimally trained persons are given, which includes a camera for obtaining images of a fundus of a subject's eye, in combination with mathematical methods to assign real time image quality classification to the images obtained based upon a set of criteria. In [US20150110372] also: Embodiments disclose systems and methods that aid in screening, diagnosis and/or monitoring of medical conditions are described. The systems and methods may allow, for example, for automated identification and localization of lesions and other anatomical structures from medical data obtained from medical imaging devices, computation of image-based biomarkers including quantification of dynamics of lesions, and/or integration with telemedicine services, programs, or software. In [US20190313895] Machine learning algorithms are applied to OCT scan image data of a patient's retina to assess various eye diseases of the patient, such as ARMD, glaucoma, and diabetic retinopathy. The classification modules for each tested-for disease or condition preferably comprises an ensemble of machine learning algorithms, preferably including both deep learning and traditional machine learning (non-deep learning) algorithms. Drawback in all three previous examples is the lack of using logical rules coupled with previous experience, as done in Case Based Reasoning, to diagnose new cases. Also: Diagnosis suggestions represent opinions of single agents, not reached via consensus and mutual agreement between more than one expert.

Known are deep learning methodologies coupled with Case Base Reasoning such as the one described in [US20190110753A1], where Deep learning algorithms enable the automated analysis of ophthalmic images to generate predictions of comparable accuracy to clinical experts. Drawback is: Diagnosis suggestions represent opinions of single agents, not reached via consensus and mutual agreement between more than one expert.

Known are Case Based Expert Systems as described for example in [Case-Based Reasoning, Introduction and Recent Developments, Ralph Bergmann, Klaus-Dieter Althoff, Mirjam Minor, Meike Reichle, Kerstin Bach]. In sum, Case Based Reasoning (CBR) is reasoning by remembering: previously solved problems (cases) are used to suggest solutions for novel but similar problems. There are four assumptions about the world around us that represent the basis of the CBR approach: 1. Regularity: the same actions executed under the same conditions will tend to have the same or similar outcomes. 2. Typicality: experiences tend to repeat themselves. 3. Consistency: small changes in the situation require merely small changes in the interpretation and in the solution. 4. Adaptability: when things repeat, the differences tend to be small, and the small differences are easy to compensate for. FIG. 2 illustrates how the assumptions listed above are used to solve problems in CBR [Introduction to Machine Learning& Case-Based Reasoning, course: 395, Computing Department, Imperial College London, Maja Pantic]. Once the currently encountered problem is described in terms of previously solved problems, the most similar solved problem can be found. The solution to this problem might be directly applicable to the current problem but, usually, some adaptation is required. The adaptation will be based upon the differences between the current problem and the problem that served to retrieve the solution. Once the solution to the new problem has been verified as correct, a link between it and the description of the problem will be created and this additional problem-solution pair (case) will be used to solve new problems in the future. Adding of new cases will improve results of a CBR system by filling the problem space more densely.

Not known are methods in which human- and machine-agents collaborate in reaching probable diagnosis and action recommendations, via an electronic voting system, in a case-based analysis manner and using simple imaging devices to take on-the-fly pictures of the retina. In addition to its main role of collecting and evaluating votes of participating agents, the voting system monitors messages, external conditions and critical parameters of connected devices, foresees critical events and selects a decision of one or more agents according to preassigned weights.

### OBJECTIVE OF THE INVENTION

The invention's goal is to increase reliability of retina disease diagnosis through facilitating collaboration between human- and machine-agents using an electronic voting system, wherein agents use Case Based Reasoning and simplified, but accurate, easy to use retina imaging devices to reach the best possible medical judgment.

### NATURE OF THE INVENTION

The invention is based on the objective of creating a method of the type mentioned above which enables human- and machine-agents to cooperate and optimize decisions related to retina diseases in the most general and complete form and in such a way that an automatic vote collection and management procedure uses, in addition to human opinion, automatic Case Based Reasoning achieving, solution proposals for newly encountered problems via previous experience and predicted information. This object is achieved by method steps described in the Patent claims 1-5.

Example of Accomplishment

I—Taking Retina Images On-the-Fly

Smartphone fundus photography is a cheap, cost effective, portable and a convenient method for retinal imaging. With practice and use of smartphone camera applications designed for this purpose, the beginners can master this technique. By training young ophthalmology residents and ophthalmic primary caretakers, this retinal imaging technique can be utilized for artificial intelligence, patient diagnostic and educational purposes [Iqbal, U. Smartphone fundus photography: a narrative review. Int J Retin Vitr 7, 44 (2021)].

Retinal imaging systems can be divided into:
Fundus cameras (mydriatic and non-mydriatic): Fundus camera can be mydriatic, which requires pupil dilation and uses bright light flash, or non-mydriatic which is more advanced and does not require pupil dilation and uses a flashlight of low intensity [Kulkarni S, Deshpande M. Recent advances in retinal imaging and diagnostics. Community Eye Health. 2019; 32(107)].
Handheld and instrument mounted cameras: Hand-held fundus camera (based on the principle of traditional fundus camera), indirect ophthalmoscope mounted fundus camera, and slit-lamp mounted camera are also used for retinal imaging. These devices are mostly high cost and are used limitedly in the routine practice of ophthalmology [Nazari Khanamiri H, Nakatsuka A, El-Annan J. Smartphone Fundus Photography. J Vis Exp. 2017].
Use of smartphones for retinal imaging: High-quality cameras and high-resolution video features of smartphones can also be utilized for retina imaging. After their initial breakthrough, there has been much advancement in the field of smartphone-based retinal imaging. Initially, smartphone fundus photography was performed by hanging it to the eyepiece of the slit lamp and focusing the retina using a 78 D or 90 D condensing lens. This method required a great deal of hand stability and was difficult [Haddock L J, Qian C. Smartphone technology for fundus photography. Retin Phys. 2015; 12(6):51-8.].

[Rajalakshmi R, Subashini R, Anjana R M, Mohan V. Automated diabetic retinopathy detection in smartphone-based fundus photography using artificial intelligence. Eye. 2018; 32(6):1138-44.] evaluated the use of artificial intelligence-based interpretation of the fundus pictures captured by using a smartphone. They reported high sensitivity for this method in the detection of diabetic retinopathy, retinal detachment, and other retinal pathologies in patients presenting at a general practitioner clinic.

Combining the above ideas: The current invention enables, through the use of stabilization devices: Helmet (FIG. 5), Simple Stand (FIG. 6) or in which the relative position of a smartphone to the retina can accurately be adjusted and stabilized, taking detailed pictures of the retina and sending them to an automatic image processing system (FIG. 7 shows an overview of an example Voting Session), which communicates all relevant image data to the connected CBR.

II—CBR Applied to Retina Disease Diagnosis

The process applied by machine-agents and underlying the current invention is based upon the idea of constructing solution pairs (cases) from collected data and performed diagnosis.

In general, three main approaches to case-based organization can be distinguished: flat organization, clustered organization, and hierarchical organization. Also, a combination of these methods within the same case base is possible. Flat organization is the simplest case-based organization that yields a straightforward flat structure of the case base. Though advantageous due to its simplicity and facile case addition/deletion, a flat case-based organization imposes, in general, case retrieval based upon a case-by-case search of the whole case base. Hence, for medium and large case bases, this leads to time-consuming retrieval, yielding an inefficient CBR system. Clustered organization, originating in the dynamic memory model initially proposed by Schank [R. C. Schank, Dynamic memory: A theory of reminding and learning in computers and people. Cambridge, UK: Cambridge University Press, 1982.], is the type of case-based organization in which cases are stored in clusters of similar cases. The grouping of cases may be based on their mutual similarity (like in the case of the dynamic memory of experiences used by Pantic [M. Pantic, Facial Expression Analysis by Computational Intelligence Techniques. PhD thesis, Delft University of Technology, 2001], [M. Pantic and L. J. M. Rothkrantz, “Case-based reasoning for user-profiled recognition of emotions from face images”, Proc. IEEE Int'l Conf. Multimedia and Expo, 2004.]) or on the similarity to some prototypical cases. The advantage of this organization is that the selection of the clusters to be matched is rather easy, as it is based upon the indexes and/or prototypical cases characterizing the clusters. A disadvantage is that it needs a more complex algorithm for case addition/deletion than a flat organized case base. Hierarchical organisation, originating in the category-exemplar memory model of Porter and Bareiss [B. W. Porter and E. R. Bareiss, “PROTOS: Experiment in knowledge acquisition for heuristic classification tasks”, Proc. 1st Int'l Meeting on Advances in Learning, pp. 159-174, 1986.], is the case-base organization that is generally obtained when cases that share the same features are grouped together. The case memory is a network structure of categories, semantic relations, cases, and index pointers. Each case is associated with a category, while the categories are inter-linked within a semantic network containing the features and intermediate states referred to by other terms. Different case features are assigned different importance in describing the membership of a case to a category. It must be noted that this importance assignment is static; if it changes, the case-based hierarchy has to be redefined. A new case is stored by searching for a matching case and by establishing the relevant feature indexes. If a case is found with only minor differences to the new case, the new case is usually not retained. In turn, a hierarchical case-based organization facilitates fast and accurate case retrieval. However, its higher complexity implies a rather cumbersome case addition/deletion, potentially involving expensive case-based reorganization and an inapt case base evaluation and maintenance. In this invention flat case-based organization is adopted.

Given a description of a problem, a retrieval algorithm should retrieve cases that are most similar to the problem or situation currently presented to the pertinent CBR system. The retrieval algorithm relies on the indices and the organization of the case memory to direct the search to case(s) potentially useful for solving the currently encountered problem. The issue of choosing the best matching cases can be referred to as analogy drawing, that is, comparing cases in order to determine the degree of similarity between them. Many retrieval algorithms have been proposed in the literature up to date: induction search (e.g., ID3, [J. R. Quinlan, Programs for Machine Learning. San Mateo, USA: Morgan Kaufmann, 1993.]), nearest neighbor search, serial search, hierarchical search, parallel search, etc. (for examples, see [T. M. Mitchell, Machine Learning. Singapore: McGraw-Hill Companies Inc., 1997.]). The simplest form of retrieval is the kst-nearest-neighbor search of the case base, which performs similarity matching on all the cases in the case base and returns just one best match [T. M. Mitchell, Machine Learning. Singapore: McGraw-Hill Companies Inc., 1997]. Nearest-neighbor retrieval is a simple approach that computes the similarity between stored cases and new input case based on weight features. A typical evaluation function is used to compute nearest-neighbor matching as shown in FIG. 3, where wᵢ is the importance weight of a feature, sim is the similarity function of features, and fᵢ^{I} and fᵢ^{R} are the values for feature i in the input and retrieved cases respectively. FIG. 4 displays a simple scheme for nearest-neighbor matching. In this 2-dimensional space, case3 is selected as the nearest neighbor because similarity(NC,case3)>similarity(NC, case1) and similarity(NC, case3)>similarity(NC, case2).

This invention uses nearest-neighbor retrieval algorithms to find cases similar to the produced, new ones.

Generally, once a matching case is retrieved, it will not correspond to exactly the same problem as the problem for which the solution is currently being sought. Consequently, the solution belonging to the retrieved case may not be optimal for the problem presently encountered and, therefore, it should be adapted. Adaptation looks for prominent differences between the retrieved case and the current case, and then (most commonly) applies a formulae or a set of rules to account for those differences when suggesting a solution. In general, there are two kinds of adaptation in CBR [I. Watson and F. Marir, “Case-based reasoning: A review”, The Knowledge Engineering Review, vol. 9, no. 4, pp. 327-354, 1994.]: 1. Structural adaptation applies adaptation rules directly to the solution stored in cases. If the solution comprises a single value or a collection of independent values, structural adaptation can include modifying certain parameters in the appropriate direction, interpolating between several retrieved cases, voting, etc. However, if there are interdependencies between the components of the solution, structural adaptation requires a thorough comprehension and a well-defined model of the problem domain. 2. Derivational adaptation reuses algorithms, methods, or rules that generated the original solution to produce a new solution to the problem currently presented to the system. Hence, derivational adaptation requires the planning sequence that begot a solution to be stored in memory along with that solution. This kind of adaptation, sometimes referred to as re-instantiation, can only be used for problem domains that are well understood. An ideal set of rules must be able to generate complete solutions from scratch, and an effective and efficient CBR system may need both structural adaptation rules to adapt poorly understood solutions and derivational mechanisms to adapt solutions of cases that are well understood. However, one should be aware that complex adaptation procedures make the system more complex but not necessarily more powerful. Complex adaptation procedures make it more difficult to build and maintain CBR systems and may also reduce system reliability and, in turn, user's confidence in the system if faulty adaptations are encountered due to, for example, incompleteness of the adaptation knowledge, which is the most difficult kind of knowledge to acquire [W. Mark, E. Simoudis and D. Hinkle, “Case-based reasoning: Expectations and results”, Case-Based Reasoning: Experiences, Lessons & Future Directions, D. B. Leake, (Ed.), pp. 269-294, AAAI Press, Menlo Park, USA, 1996.]. Therefore, in many CBR systems, adaptation is done by the user rather than by the system. Mark et al. report that in a well-designed system, the users do not perceive “manual” adaptation as something negative [W. Mark, E. Simoudis and D. Hinkle, “Case-based reasoning: Expectations and results”, Case-Based Reasoning: Experiences, Lessons & Future Directions, D. B. Leake, (Ed.), pp. 269-294, AAAI Press, Menlo Park, USA, 1996.]. This invention adopts a structural adaptation algorithm using simple parameter-value substitution as shall be seen below.

Formalism

An expert system of the kind this invention is adopting may be best described and formulated in mathematical logics using horn programs (see [Gupta, G. (1999). Horn Logic Denotations and Their Applications. In: Apt, K. R., Marek, V. W., Truszczynski, M., Warren, D. S. (eds) The Logic Programming Paradigm. Artificial Intelligence. Springer, Berlin, Heidelberg] for a quick introduction). The following definitions serve in doing that.

Definition-1: Horn Clauses

A clause (i.e., a disjunction of occurrences of variables which are called literals) is called a Horn clause if it contains at most one positive literal. Horn clauses are usually written as

L₁, . . . ,Lₙ⇒L(≡¬L₁∨ . . . ∨¬Lₙ∨L)

or

L₁, . . . ,Lₙ⇒(≡¬L₁∨ . . . ∨¬Lₙ),

where n>=0 and L is the only positive literal. A definite clause is a Horn clause that has exactly one positive literal. A Horn clause without a positive literal is called a goal. Horn clauses express a subset of statements of first-order logic. The programming language Prolog is built on top of Horn clauses. Prolog programs are comprised of definite clauses and any question in Prolog is a goal.

Definition-2: SLD (Selection Function in Linear Resolution for Definite Clauses) Resolution

An SLD derivation of Cₘ from a set {C₁, . . . , Cₙ} of Horn clauses (with the non-negated literal in the first place, if it exists) is a sequence C₁, . . . , Cᵢ, . . . , Cₙ, Cₙ₊₁, . . . , Cₘ such that Cₙ₊₁ is the resolvent of Cᵢ (goal clause) and another C∈{C₁, . . . , Cₙ} for every j>n+1, Cⱼ is the resolvent of Cⱼ₋₁ and another C∈{C₁, . . . , Cₙ}. Every resolution step takes the form:

L′∨C′,¬L″∨C″=>(C′∨C″)(MGU(L′,L″))

Where an MGU is an assignment of truth values for literals in L′ and L″ which renders them syntactically equal. SLD resolution is complete for Horn clauses: A set of Horn clauses is unsatisfiable iff there exists an SLD refutation for it. This, as well as efficiency considerations, makes SLD resolution one of the safest ways to perform deductive queries. An Example illustrating SLD Resolution of Horn Clauses is the following:

Let the knowledge-base be KB={ActionRecommendation, A⊃B, A∧C⊃D, C⊃M, M∧B⊃ActionRecommendation, NoRecommendation}, the Goal G={ActionRecommendation}, then the SLD refutation sequence looks like this:

```
{¬ ActionRecommendation }=>
  { ¬A, ¬C}=>
    {¬D}=>
    {¬B}=>
      [ ]
```

Definition-3: CBR cases

CBR cases are definite clauses wherein the body is called Problem and the unique, positive literal is called Solution. Any predicate, expressed in one or more clauses, in the body is called a Feature. CBR Case Knowledge bases are sets of definite clauses also called Programs.

Definition -4: Nearest neighbor retrieval algorithm
Algorithm 1

Inputs: KB, new sought CBR case (C), set of weights of all features used in

all CBR cases

| Output: Set of pair-wise similar CBR cases | | |
|---|---|---|
| Body: Set MaxSim= 0, SelectedCase = First Case in KB | | |
| | For all CBR cases C': | |
| | - | Calculate MaxSim=similarity(C,C') using the formula |
| | | given in Figure 3 |
| | - | If the calculated MaxSim is higher than the one stored, |
| | | replace the one stored and the SelectedCase with C' |

ReturnSelectedCase

Definition - 5: Parameter-value substitution algorithm
Algorithm 2

```
  Inputs: CBR case (C) , Set of Literal mappings of the form
M={{L₁>L₂}...{Lᵢ>Lⱼ}}
  Output: Modified CBR case
  Body: For all literals in C:
    If {Lₓ>L_{y}} is element of M: Replace Lₓ with L_{y} in C
```

Finally, an example illustrates best the accomplishment of this invention (illustrated in FIG. 7).

III—Diagnosing Retinopathy of Prematurity (ROP) or Retinal Detachment (RD)

Goal is the automatic recognition of either ROP or RD and reaching, via an electronic voting system, an adequate diagnosis for incoming new cases. A machine-agent uses the following consequent steps for this purpose:
1. Look for signs of age of the retina
2. Look for retina signs generally
3. Determine certainty of present signs
4. Determine diseases linked to those signs and their weights
5. Calculate a score for every disease possibility
6. Determine the most possible diagnosis depending on previous cases scores and their diagnosis
7. Store the new case in the cases database

1. Look for Retina Signs of Age:

Referring to image processing
1—Tilted optic disc A Greyish Color A Dark Ring Surround⊃neonate optic disc
2—Less well-developed fovea⊃neonate fovea
3—High density Vasculature⊃neonate vasculature
4—neonate optic disc A neonate fovea A neonate vasculature⊃neonate retina

If any rule applies then: neonate retina else adult retina

2. Look for Retina Signs Generally:

Referring to image processing
Sign 1:—Line separate vasculature from no vasculature A line diameter less than 0.5∧line color is white⊃Stage 1 ROP
Sign 2—Line separate vasculature from no vasculature A line diameter more than 0.5∧line color is white⊃Stage 2 ROP
Sign 3—Line separate vasculature from no vasculature A line diameter more than 0.5∧line color is red⊃Stage 3 ROP
Sign 4—Patch of no vasculature A Folds/wrinkles A no normal vasculature within the patch⊃Partial RD
Sign 5—Patch of no vasculature A Folds/wrinkles A No normal vasculature at all >Complete RD
Sign 6—Area of abnormality within optic disc and macula⊃Zone 1
Sign 7—Area of abnormality at distance twice the Optic disc/macula distance⊃Zone 2
Sign 8—Area of abnormality all over the retina⊃Zone 3
Sign 9—Abnormal Vasculature within optic disc⊃Plus Disease

3. Determine Certainty of Present Signs:

Referring to image processing
Sign 1:90%
Sign 2: 70%
Sign 3: 85%
Sign 4: 95%
Sign 5: 90%
Sign 6: 70%
Sign 7: 90%
Sign 8: 70% . . . etc

4. Determine Diseases Linked to Those Signs and their Weights

Referring to the medical database
Sign1—ROP
Sign2—ROP
Sign3—ROP
Sign4—ROP, RD
Sign5—ROP, RD

5. Calculate a Score for Every Disease Possibility:

Referring to the medical database:

ROP:
Stage 1: 2 points,
Stage 2: 2 points,
Stage 3: 2 points,
Stage 4: 6 points,
Stage 5: 6 points,
Plus Disease: 5 points,
Zone 1: 4 points,
Zone 2: 3 points,
Zone 3: 1 points,

RD:
Partial RD: 60 points,
Complete RD: 100 points,

Calculation For ROP:
Sign 1: percentage×its weight in ROP “2”
Sign 2: percentage×its weight in ROP “2”
Sign 3: percentage×its weight in ROP “2”
Sign 4: percentage×its weight in ROP “6”
Sign 5: percentage×its weight in ROP “6”
Sign 6: percentage×its weight in ROP “4”
Sign 7: percentage×its weight in ROP “3”
Sign 8: percentage×its weight in ROP “1”
Sign 9: percentage×its weight in ROP “5”

Calculation For RD:
Sign 4: percentage×its weight in RD “60”
Sign 5: percentage×its weight in RD “100”

6. Determine the Most Possible Diagnosis Depending on Previous Cases Scores and their Diagnosis

Following is an example CBR process assuming the existence of a Case Database as well as the possibility of human intervention to modify diagnosis of cases after the automated process reaches a suggested conclusion:

Case Database

Case 1

3 weeks old baby, born at 1570 gm, after 37 weeks.

He was exposed to 100% oxygen for 3 days.

Image Signs:
1. 1st sign: Stage: 2, Stage Percentage: 80%
2. 2nd sign: Zone: 2, Zone Percentage: 60%
3. 3rd sign: Plus disease: Yes, Plus Percentage: 80%

Diagnosis: ROP

Overall score: 0.8*2+0.6*3+0.8*5=7.4

Case 2

4 weeks old baby, born at 1820 gm, after 29 weeks.

He was exposed to 100% oxygen for 1 week days

Image Signs:
1. 1st sign: Stage 1, Percentage: 75%
2. 2nd Sign: Zone 3, Percentage: 80%
3. 3rd Sign: Plus disease: No, Percentage: 0%

Diagnosis: ROP

Overall score: 0.75*2+0.8*1+0*5=2.3

Case 3

13 days old baby, born at 2100 gm, after 34 weeks

He had respiratory distress and was exposed to 100% oxygen for 5 days

Image Signs:
1. 1st sign: Stage 1, Percentage: 90%
2. 2nd Sign: Zone 2, Percentage: 60%
3. Plus disease: Yes, Percentage: 40%

Diagnosis: ROP

Overall score: 0.9*2+0.6*3+0.4*5=5.6

Case 4

23 days old baby, born at 1750 gm, after 34 weeks

Delivered normally

Image Signs:
1. 1st Sign: Stage 3, Stage Percentage: 80%
2. 2nd Sign: Zone: 2, Zone Percentage: 85%
3. 3rd Sign: Plus disease: Yes, Plus Percentage: 45%

Diagnosis: ROP

Overall score: 0.8*4+0.85*6+0.45*10=12.8

Case 5

25 days old baby, born at 1950 gm, after 34 weeks

He was born with a defect in his heart. Has been on 100% oxygen since birth.

Image Signs:
1. 1st Sign: Partial Retinal Detachment, Percentage: 75%
2. 2nd Sign: Plus Disease, Percentage: 15%

Diagnosis: RD

Overall score: 0.75*60+0.15*5=45.75

New Case 1:

2 weeks old baby, born at 1980 gm, after 33 weeks

He had respiratory distress and was put on ventilation with 100% oxygen since birth

Image Signs:
1. 1st sign: Stage 2, Percentage: 60%
2. 2nd sign: Zone 1, Percentage: 90%
3. 3rd sign: Plus disease: YES, Percentage: 80%
4. 4th sign: Partial Retinal Detachment, Percentage: 10%

Two Options:

ROP Overall score: 0.6*2+0.9*4+0.8*5=8.8

RD Overall score: 0.1×60=6

Case 1 is the nearest, Diagnosis: ROP

New Case 2

20 days old baby, born at 2980 gm, after 31 weeks

He had respiratory distress for 2 days

Image Signs:
1. 1st sign: Partial Retinal Detachment, Percentage: 85%
2. 2nd sign: Plus disease: YES, Percentage: 20%

Two Options:

ROP Overall score: 0.2*5=1

RD Overall score: 0.85×60=51

Case 5 is the nearest, Diagnosis: RD

7. Store the New Case in the Cases Database

The following two cases are added to the case database:

Case 6

2 weeks old baby, born at 1980 gm, after 33 weeks

He had respiratory distress and was put on ventilation with 100% oxygen since birth

Image Signs:
5. 1st sign: Stage 2, Percentage: 60%
6. 2nd sign: Zone 1, Percentage: 90%
7. 3rd sign: Plus disease: YES, Percentage: 80%
8. 4th sign: Partial Retinal Detachment, Percentage: 10%

Diagnosis: ROP

Overall score: 0.6*2+0.9*4+0.8*5=8.8

Case 7

20 days old baby, born at 2980 gm, after 31 weeks

He had respiratory distress for 2 days

Image Signs:
3. 1st sign: Partial Retinal Detachment, Percentage: 85%
4. 2nd sign: Plus disease: YES, Percentage: 20%

Diagnosis: RD

Overall score: 0.85×60=51

IV—Reaching a Final Diagnosis Through Voting

A marking feature of the current invention is enabling cooperation between human- and machine-agents and raising quality and reliability of the final diagnosis through electronic voting.

For this purpose, two sub-systems, the ‘Voting Manager’ and the ‘Individual Voter’ communicate within a ‘Voting Session’ to reach a final voting result. Every Voting Session comprises a ‘Voting Policy’, determined by an agent, who chooses to be a Voting Manager. A human agent can be in the same time Voting Manager and Individual Voter. An Individual Voter can propose a diagnosis and request the initiation of a Voting Session from the Voting Manager. The Voting Policy includes, but is not limited to:
1—Voting Weights marking the difference between human- and machine votes (default is: Weight-of-human-vote=3×Weight-of-machine-vote)
2—Minimal number of machine- and human voters (default is: 2)
3—Pre-Voting Session duration (i.e. the time it takes an agent to verify correctness of a proposed diagnosis)
4—Allowed number of repetitions of the Session

Definition -6: Voting Manager Algorithm

Inputs: Proposed Diagnosis, Request for the initiation of a Voting Session,

Electronic addresses of participants

Output: Voting Result, Request for Repetition or Request for Cancellation
Body:

| - | Send Voting Session Initiation Signal to all | | |
|---|---|---|---|
| | participants | | |
| - | Send Voting Session Policy and Proposed Diagnosis to | | |
| | all participants | | |
| - | Wait until: | | |
| | | ○ | All Voting Signals are received from |
| | | | Participants |
| | | ○ | Any Participant requests repetition |
| | | ○ | Any Participant requests Cancellation |

| | |
|---|---|
| - | If all Voting Signals received and no cancellation or |
| | repetition requests exist: Calculate Voting Result using |
| | Session Policy |

Return Voting Result, Request for Repetitionor Request

for Cancellation

Definition -7: Individual Voter Algorithm

Inputs: Proposed Diagnosis, Voting Session Signal, Voting Policy
Output: Individual decision, Request for Repetition or Request for

Cancellation

| Body: | | | | | | | | | |
|---|---|---|---|---|---|---|---|---|---|
| | - | Read Voting Policy and determine pre-voting duration | | | | | | | |
| | - | For the pre-voting duration: | | | | | | | |
| | | | ○ | Use available image-processing, medical | | | | | |
| | | | | database and local database information to | | | | | |
| | | | | reach a probable diagnosis | | | | | |
| | | | o | If a probable diagnosis is reached within pre- | | | | | |
| | | | | voting duration, then: | | | | | |
| | | | | | ▪ | Compare probable diagnosis with | | | |
| | | | | | | proposed diagnosis | | | |
| | | | | | | | • Return | | |
| | | | | | | | | ○ | Individual Vote = true, if |
| | | | | | | | | | probable |
| | | | | | | | | | diagnosis=proposed |
| | | | | | | | | | diagnosis, otherwise |
| | | | | | | | | | Individual Vote =false |

| - | If number of repetitions is reached | | | | |
|---|---|---|---|---|---|
| | | ○ | Return Request for Cancellation | | |
| | | | | ▪ | Else |

• Return Request for Repetition

### DESCRIPTION OF THE DRAWINGS

The present invention may be better understood, and its numerous features and advantages made apparent to those skilled in the art by referencing the accompanying drawings.

FIG. 1 Communications to and from an agent with Voting Manager;

FIG. 2 CBR Way of Work;

FIG. 3A nearest-neighbor evaluation function;

FIG. 4 How to find the nearest neighbor of the new case NC;

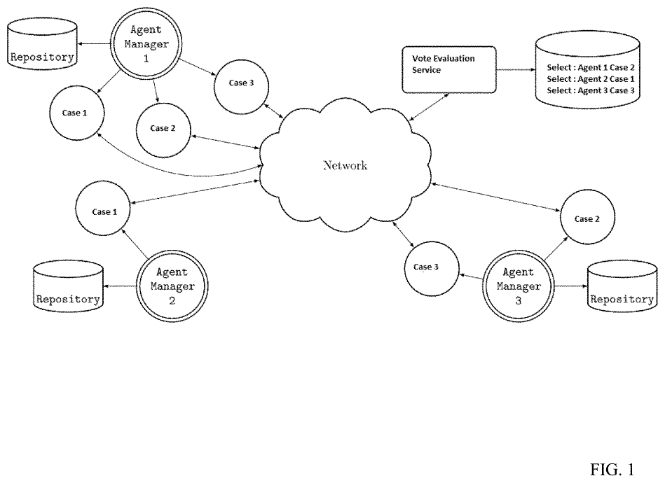

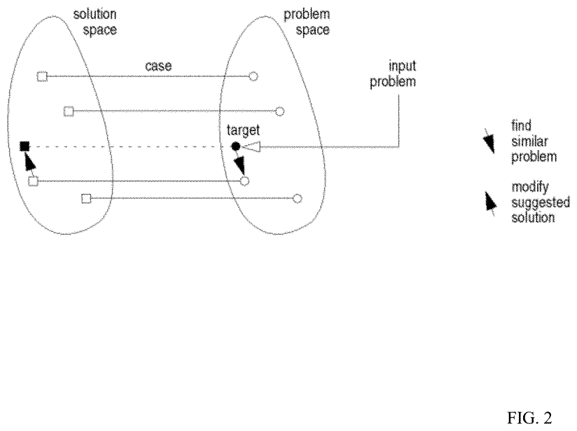

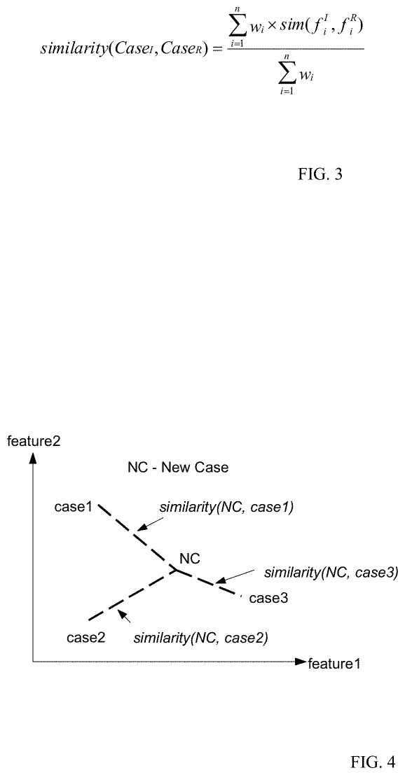

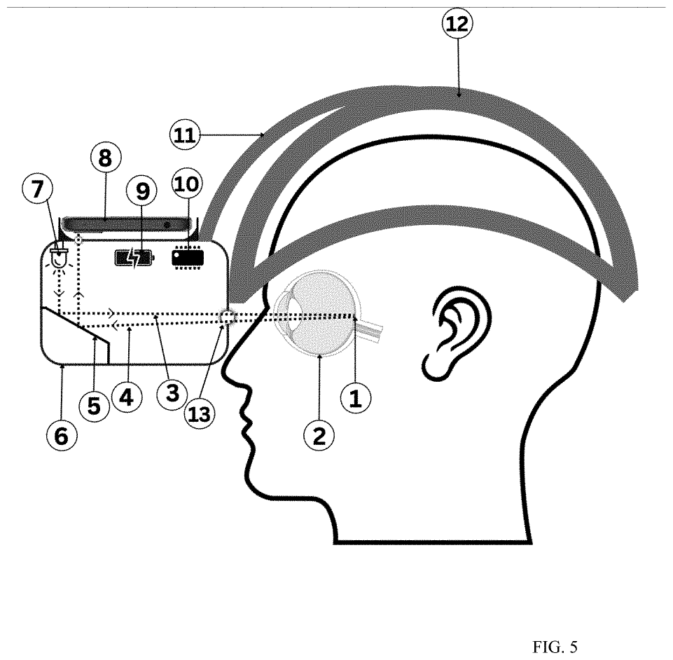

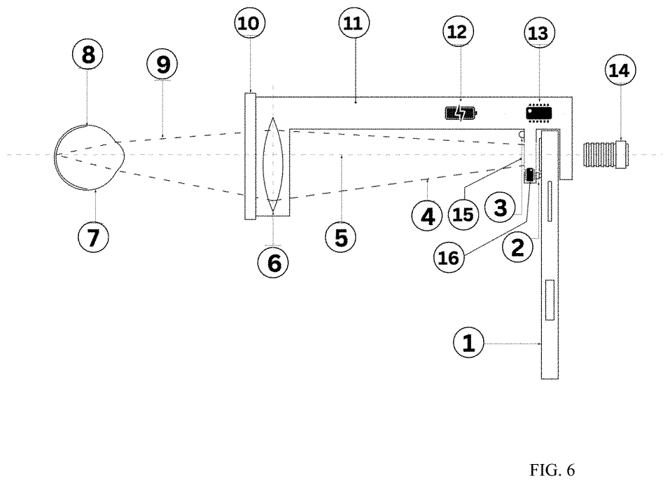

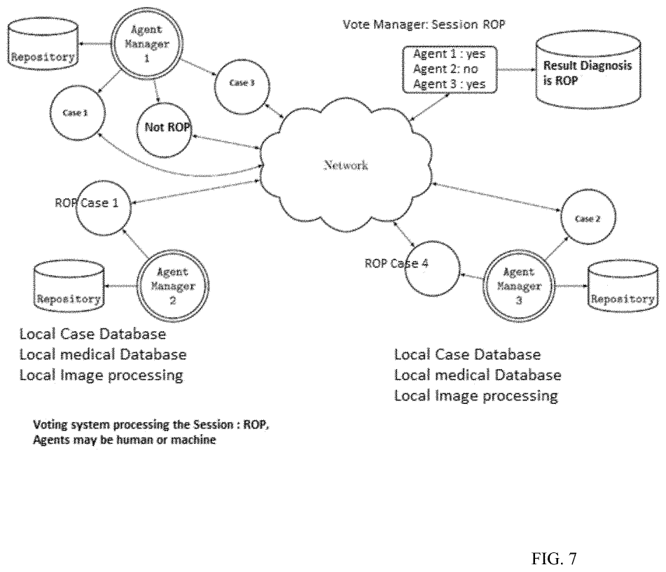

FIG. 5 Helmet
1. Viewed spot of retina
2. Eye ball
3. Outgoing light
4. Reflected light
5. Mirror/prism mirror
6. Holding frame
7. Light source
8. Smartphone
9. Battery
10. Electronic module for battery charging and other functions
11. Holding belt
12. Helmet
13. Opening for emerging and reflected light;

FIG. 6 Simple Stand
1. Smartphone
2. ON/OFF Switch for light source
3. Light source
4. Outgoing/reflected light
5. Median plane
6. Lens power range 12 D to 40 D
7. Eye ball
8. Retina
9. Outgoing/reflected light
10. Lens Cap
11. Device Body
12. Battery
13. Electronic module for battery charging and other functions
14. Bolt
15. opening for smartphone camera
16. Electronic module for light source;

FIG. 7 Example Voting Session.

1. A method to reach a diagnosis through electronic voting, involving both human- as well as machine agents in a multi-agent environment, the method comprising steps of:
a. Determining Proposed Diagnosis, Voting Manager, Voting Session, and Voting policy, the latter including: Voting Weights, Minimal number of machine- and human voters, Pre-Voting Session duration and allowed number of repetitions of the Session
b. Sending Voting Session initiation signals to all participants
c. Waiting until agent individual votes are determined and sent via Individual Voter procedures to the Voting Manager
d. Reaching a final decision comprising one of: The proposed diagnosis, a vote repetition request, a vote cancellation request

2. The method of claim 1, wherein Individual Voter procedures examine diagnosis suggestions of human- or machine-agents, supported by image processing-, Case Based Reasoning- and medical database systems related to retina diseases, the method comprising the steps of:
a. Participating in a Voting Session initiated by a Voting Manager according to a given Voting Policy and concerning a given proposed diagnosis
b. Using special-purpose stabilization devices to enhance quality of retina images captured via smart-phones in pre-voting sessions
c. Sharing all relevant data of the examined case, including image-processing information, with participants of a Voting Session and providing them access to locally held Case Based Reasoning databases
d. Using locally held image-processing- and medical database systems as well as shared information of other participants of a Voting Session to deduce symptoms/signs of possible diseases as well as weights per disease
e. Using examined case data to calculate an overall score and compare this score to locally held as well as shared scores of old Case Based Reasoning cases, finding the most probable diagnosis and logging the new case into a local cases database
f. Comparing the most probable diagnosis with the one suggested in the Voting Session and automatically sending either: yes' or ‘no’ to the Voting Manager.

3. The method of claim 2, wherein Case Based Reasoning uses horn programs to model cases and case data

4. A system to reach a diagnosis through electronic voting, involving both human- as well as machine agents in a multi-agent environment, said system comprising:
a. Sub-systems determining Proposed Diagnosis, Voting Manager, Voting Session, and Voting policy, the latter including: Voting Weights, Minimal number of machine- and human voters, Pre-Voting Session duration and allowed number of repetitions of the Session
b. Sub-systems sending Voting Session initiation signals to all participants
c. Sub-systems waiting until agent individual votes are determined and sent via Individual Voter procedures to the Voting Manager
d. Sub-systems reaching a final decision comprising one of: The proposed diagnosis, a vote repetition request, a vote cancellation request

5. The sub-system of claim 4, wherein Individual Voter sub-system examines diagnosis suggestions of human- or machine-agents, supported by image processing-, Case Based Reasoning- and medical database systems related to retina diseases, the said sub-system comprising:
a. Sub-systems participating in a Voting Session initiated by a Voting Manager according to a given Voting Policy and concerning a given proposed diagnosis
b. Sub-systems using special-purpose stabilization devices to enhance quality of retina images captured via smart-phones in pre-voting sessions
c. Sub-systems sharing all relevant data of the examined case, including image-processing information, with participants of a Voting Session and providing them access to locally held Case Based Reasoning databases
d. Sub-systems using locally held image-processing- and medical database systems as well as shared information of other participants of a Voting Session to deduce symptoms/signs of possible diseases as well as weights per disease
e. Sub-systems using examined case data to calculate an overall score and compare this score to locally held as well as shared scores of old Case Based Reasoning cases, finding the most probable diagnosis and logging the new case into a local cases database
f. Sub-systems comparing the most probable diagnosis with the one suggested in the Voting Session and automatically sending either: ‘yes’ or ‘no’ to the Voting Manager.
